(19) United States
(12) Patent Application Publication
(10) Pub. No.: US 2005/0049797 A1
(43) Pub. Date: Mar. 3, 2005

(54) Method and system for displacement-vector-based detection of zone misalignment in microarray data

(76) Inventors: Srinka Ghosh, San Francisco, CA (US); Nicholas M. Sampas, San Jose, CA (US)

Correspondence Address: AGILENT TECHNOLOGIES, INC.;Legal Department, DL429, Intellectual Property Administration, P.O. Box 7599, Loveland, CO, 80537-0599, US

(21) Appl. No.: 10/650,139

(22) Filed: Aug. 27, 2003

(51) Int. Cl. G01N 31/00; G01N 33/50; G01N 33/48; G06F 19/00

(52) U.S. Cl. 702/22; 702/19

(57) ABSTRACT

A method and system for detecting block and zone misalignment of feature positions within a microarray-data set and for correcting feature positions for block or zone misalignment. In one embodiment of the present invention, displacement vectors representing the vector differences between observed positions of features and expected positions for the features of a microarray are calculated, based on an initially determined coordinate system. Features within a microarray data set are then partitioned with respect to the calculated vector displacements, so that features misaligned by a common rotation or translation are partitioned into a separate partition. A correction for each common misalignment can then be calculated and applied to the features of each partition.

### TECHNICAL FIELD

The present invention is related to processing of microarray data and, in particular, to a method and system for partitioning microarray data based on displacement vectors calculated for feature positions so that the partitions represent blocks or zones of features that are commonly aligned.

### BACKGROUND OF THE INVENTION

The present invention is related to one of a number of initial steps in processing microarray data concerned with identifying, as accurately as possible, the positions of features on the two-dimensional surface of the microarray. A general background of microarray technology is first provided, in this section, to facilitate discussion of microarray-data processing, in following subsections. It should be noted that microarrays are also referred to as “microarrays” and simply as “arrays.” These alternate terms may be used interchangeably in the context of microarrays and microarray technologies. Art described in this section is not admitted to be prior art to this application.

Array technologies have gained prominence in biological research and are likely to become important and widely used diagnostic tools in the healthcare industry. Currently, microarray techniques are most often used to determine the concentrations of particular nucleic-acid polymers in complex sample solutions. Molecular-array-based analytical techniques are not, however, restricted to analysis of nucleic acid solutions, but may be employed to analyze complex solutions of any type of molecule that can be optically or radiometrically scanned and that can bind with high specificity to complementary molecules synthesized within, or bound to, discrete features on the surface of an array. Because arrays are widely used for analysis of nucleic acid samples, the following background information on arrays is introduced in the context of analysis of nucleic acid solutions following a brief background of nucleic acid chemistry.

Deoxyribonucleic acid (“DNA”) and ribonucleic acid (“RNA”) are linear polymers, each synthesized from four different types of subunit molecules. The subunit molecules for DNA include: (1) deoxy-adenosine, abbreviated “A,” a purine nucleoside; (2) deoxy-thymidine, abbreviated “T,” a pyrimidine nucleoside; (3) deoxy-cytosine, abbreviated “C,” a pyrimidine nucleoside; and (4) deoxy-guanosine, abbreviated “G,” a purine nucleoside. The subunit molecules for RNA include: (1) adenosine, abbreviated “A,” a purine nucleoside; (2) uracil, abbreviated “U,” a pyrimidine nucleoside; (3) cytosine, abbreviated “C,” a pyrimidine nucleoside; and (4) guanosine, abbreviated “G,” a purine nucleoside. FIG. 1 illustrates a short DNA polymer 100, called an oligomer, composed of the following subunits: (1) deoxy-adenosine 102; (2) deoxy-thymidine 104; (3) deoxy-cytosine 106; and (4) deoxy-guanosine 108. When phosphorylated, subunits of DNA and RNA molecules are called “nucleotides” and are linked together through phosphodiester bonds 110-115 to form DNA and RNA polymers. A linear DNA molecule, such as the oligomer shown in FIG. 1, has a 5′ end 118 and a 3′ end 120. A DNA polymer can be chemically characterized by writing, in sequence from the 5′ end to the 3′ end, the single letter abbreviations for the nucleotide subunits that together compose the DNA polymer. For example, the oligomer 100 shown in FIG. 1 can be chemically represented as “ATCG.” A DNA nucleotide comprises a purine or pyrimidine base (e.g. adenine 122 of the deoxy-adenylate nucleotide 102), a deoxy-ribose sugar (e.g. deoxy-ribose 124 of the deoxy-adenylate nucleotide 102), and a phosphate group (e.g. phosphate 126) that links one nucleotide to another nucleotide in the DNA polymer. In RNA polymers, the nucleotides contain ribose sugars rather than deoxy-ribose sugars. In ribose, a hydroxyl group takes the place of the 2′ hydrogen 128 in a DNA nucleotide. RNA polymers contain uridine nucleosides rather than the deoxy-thymidine nucleosides contained in DNA. The pyrimidine base uracil lacks a methyl group (130 in FIG. 1) contained in the pyrimidine base thymine of deoxy-thymidine.

The DNA polymers that contain the organization information for living organisms occur in the nuclei of cells in pairs, forming double-stranded DNA helixes. One polymer of the pair is laid out in a 5′ to 3′ direction, and the other polymer of the pair is laid out in a 3′ to 5′ direction. The two DNA polymers in a double-stranded DNA helix are therefore described as being anti-parallel. The two DNA polymers, or strands, within a double-stranded DNA helix are bound to each other through attractive forces including hydrophobic interactions between stacked purine and pyrimidine bases and hydrogen bonding between purine and pyrimidine bases, the attractive forces emphasized by conformational constraints of DNA polymers. Because of a number of chemical and topographic constraints, double-stranded DNA helices are most stable when deoxy-adenylate subunits of one strand hydrogen bond to deoxy-thymidylate subunits of the other strand, and deoxy-guanylate subunits of one strand hydrogen bond to corresponding deoxy-cytidilate subunits of the other strand.

FIGS. 2A-B illustrates the hydrogen bonding between the purine and pyrimidine bases of two anti-parallel DNA strands. FIG. 2A shows hydrogen bonding between adenine and thymine bases of corresponding adenosine and thymidine subunits, and FIG. 2B shows hydrogen bonding between guanine and cytosine bases of corresponding guanosine and cytosine subunits. Note that there are two hydrogen bonds 202 and 203 in the adenine/thymine base pair, and three hydrogen bonds 204-206 in the guanosine/cytosine base pair, as a result of which GC base pairs contribute greater thermodynamic stability to DNA duplexes than AT base pairs. AT and GC base pairs, illustrated in FIGS. 2A-B, are known as Watson-Crick (“WC”) base pairs.

Two DNA strands linked together by hydrogen bonds forms the familiar helix structure of a double-stranded DNA helix. FIG. 3 illustrates a short section of a DNA double helix 300 comprising a first strand 302 and a second, anti-parallel strand 304. The ribbon-like strands in FIG. 3 represent the deoxyribose and phosphate backbones of the two anti-parallel strands, with hydrogen-bonding purine and pyrimidine base pairs, such as base pair 306, interconnecting the two strands. Deoxy-guanylate subunits of one strand are generally paired with deoxy-cytidilate subunits from the other strand, and deoxy-thymidilate subunits in one strand are generally paired with deoxy-adenylate subunits from the other strand. However, non-WC base pairings may occur within double-stranded DNA.

Double-stranded DNA may be denatured, or converted into single stranded DNA, by changing the ionic strength of the solution containing the double-stranded DNA or by raising the temperature of the solution. Single-stranded DNA polymers may be renatured, or converted back into DNA duplexes, by reversing the denaturing conditions, for example by lowering the temperature of the solution containing complementary single-stranded DNA polymers. During renaturing or hybridization, complementary bases of anti-parallel DNA strands form WC base pairs in a cooperative fashion, leading to reannealing of the DNA duplex. Strictly A-T and G-C complementarity between anti-parallel polymers leads to the greatest thermodynamic stability, but partial complementarity including non-WC base pairing may also occur to produce relatively stable associations between partially-complementary polymers. In general, the longer the regions of consecutive WC base pairing between two nucleic acid polymers, the greater the stability of hybridization between the two polymers under renaturing conditions.

The ability to denature and renature double-stranded DNA has led to the development of many extremely powerful and discriminating assay technologies for identifying the presence of DNA and RNA polymers having particular base sequences or containing particular base subsequences within complex mixtures of different nucleic acid polymers, other biopolymers, and inorganic and organic chemical compounds. One such methodology is the array-based hybridization assay. FIGS. 4-7 illustrate the principle of the array-based hybridization assay. An array (402 in FIG. 4) comprises a substrate upon which a regular pattern of features is prepared by various manufacturing processes. The array 402 in FIG. 4, and in subsequent FIGS. 5-7, has a grid-like 2-dimensional pattern of square features, such as feature 404 shown in the upper left-hand corner of the array. Each feature of the array contains a large number of identical oligonucleotides covalently bound to the surface of the feature. These bound oligonucleotides are known as probes. In general, chemically distinct probes are bound to the different features of an array, so that each feature corresponds to a particular nucleotide sequence. In FIGS. 4-6, the principle of array-based hybridization assays is illustrated with respect to the single feature 404 to which a number of identical probes 405-409 are bound. In practice, each feature of the array contains a high density of such probes but, for the sake of clarity, only a subset of these are shown in FIGS. 4-6.

Once an array has been prepared, the array may be exposed to a sample solution of target DNA or RNA molecules (410-413 in FIG. 4) labeled with fluorophores, chemiluminescent compounds, or radioactive atoms 415-418. Labeled target DNA or RNA hybridizes through base pairing interactions to the complementary probe DNA, synthesized on the surface of the array. FIG. 5 shows a number of such target molecules 502-504 hybridized to complementary probes 505-507, which are in turn bound to the surface of the array 402. Targets, such as labeled DNA molecules 508 and 509, that do not contains nucleotide sequences complementary to any of the probes bound to array surface do not hybridize to generate stable duplexes and, as a result, tend to remain in solution. The sample solution is then rinsed from the surface of the array, washing away any unbound-labeled DNA molecules. In other embodiments, unlabeled target sample is allowed to hybridize with the array first. Typically, such a target sample has been modified with a chemical moiety that will react with a second chemical moiety in subsequent steps. Then, either before or after a wash step, a solution containing the second chemical moiety bound to a label is reacted with the target on the array. After washing, the array is ready for scanning. Biotin and avidin represent an example of a pair of chemical moieties that can be utilized for such steps.

Finally, as shown in FIG. 6, the bound labeled DNA molecules are detected via optical or radiometric scanning. Optical scanning involves exciting labels of bound labeled DNA molecules with electromagnetic radiation of appropriate frequency and detecting fluorescent emissions from the labels, or detecting light emitted from chemiluminescent labels. When radioisotope labels are employed, radiometric scanning can be used to detect the signal emitted from the hybridized features. Additional types of signals are also possible, including electrical signals generated by electrical properties of bound target molecules, magnetic properties of bound target molecules, and other such physical properties of bound target molecules that can produce a detectable signal. Optical, radiometric, or other types of scanning produce an analog or digital representation of the array as shown in FIG. 7, with features to which labeled target molecules are hybridized similar to 706 optically or digitally differentiated from those features to which no labeled DNA molecules are bound. In other words, the analog or digital representation of a scanned array displays positive signals for features to which labeled DNA molecules are hybridized and displays negative features to which no, or an undetectably small number of, labeled DNA molecules are bound. Features displaying positive signals in the analog or digital representation indicate the presence of DNA molecules with complementary nucleotide sequences in the original sample solution. Moreover, the signal intensity produced by a feature is generally related to the amount of labeled DNA bound to the feature, in turn related to the concentration, in the sample to which the array was exposed, of labeled DNA complementary to the oligonucleotide within the feature.

One, two, or more than two data subsets within a data set can be obtained from a single microarray by scanning the microarray for one, two or more than two types of signals. Two or more data subsets can also be obtained by combining data from two different arrays. When optical scanning is used to detect fluorescent or chemiluminescent emission from chromophore labels, a first set of signals, or data subset, may be generated by scanning the microarray at a first optical wavelength, a second set of signals, or data subset, may be generated by scanning the microarray at a second optical wavelength, and additional sets of signals may be generated by scanning the molecular at additional optical wavelengths. Different signals may be obtained from a microarray by radiometric scanning to detect radioactive emissions one, two, or more than two different energy levels. Target molecules may be labeled with either a first chromophore that emits light at a first wavelength, or a second chromophore that emits light at a second wavelength. Following hybridization, the microarray can be scanned at the first wavelength to detect target molecules, labeled with the first chromophore, hybridized to features of the microarray, and can then be scanned at the second wavelength to detect target molecules, labeled with the second chromophore, hybridized to the features of the microarray. In one common microarray system, the first chromophore emits light at a red visible-light wavelength, and the second chromophore emits light at a green, visible-light wavelength. The data set obtained from scanning the microarray at the red wavelength is referred to as the “red signal,” and the data set obtained from scanning the microarray at the green wavelength is referred to as the “green signal.” While it is common to use one or two different chromophores, it is possible to use one, three, four, or more than four different chromophores and to scan a microarray at one, three, four, or more than four wavelengths to produce one, three, four, or more than four data sets.

When a microarray is scanned, data may be collected as a two-dimensional digital image of the microarray, each pixel of which represents the intensity of phosphorescent, fluorescent, chemiluminescent, or radioactive emission from an area of the microarray corresponding to the pixel. A microarray data set may comprise a two-dimensional image or a list of numerical, alphanumerical pixel intensities, or any of many other computer-readable data sets. An initial series of steps employed in processing scanned, digital microarray images includes constructing a regular coordinate system for the digital image of the microarray by which the features within the digital image of the microarray can be indexed and located. For example, when the features are laid out in a periodic, rectilinear pattern, a rectilinear coordinate system is commonly constructed so that the positions of the centers of features lie as closely as possible to intersections between horizontal and vertical gridlines of the rectilinear coordinate system. Then, regions of interest (“ROIs”) are computed, based on the initially estimated positions of the features in the coordinate grid, and centroids for the ROIs are computed in order to refine the positions of the features. Once the position of a feature is refined, feature pixels can be differentiated from background pixels within the ROI, and the signal corresponding to the feature can then be computed by integrating the intensity over the feature pixels.

In general, microarrays are manufactured with the intent of positioning features as exactly periodically and regularly spaced as possible. Accurately positioning features on the surface of the microarray greatly facilitates extracting data from a scanned, digital image of a microarray produced by a microarray scanner. However, despite great care and attention paid to accurately positioning features onto the surface of microarrays during microarray manufacture, indications of feature-position errors are observed in microarray data-processing steps. Thus, designers, manufactures, and users of microarrays have recognized the need for methods for detecting and accounting for feature-position errors in microarray data.

### SUMMARY OF THE INVENTION

One embodiment of the present invention provides a method and system for detecting block and zone misalignments of feature positions within a microarray-data set and for correcting feature positions for block or zone misalignment. In a described embodiment of the present invention, displacement vectors representing the vector differences between observed positions of features and expected positions for the features of a microarray are calculated, based on an initially determined coordinate system. Features within a microarray data set are then partitioned with respect to the calculated vector displacements, so that features misaligned by a common rotation or translation are partitioned into a separate partition. A correction for the common misalignment of the features of each partition can then be calculated and applied to the features of the partition.

### BRIEF DESCRIPTION OF THE DRAWINGS

FIG. 1 illustrates a short DNA polymer 100, called an oligomer, composed of the following subunits: (1) deoxy-adenosine 102; (2) deoxy-thymidine 104; (3) deoxy-cytosine 106; and (4) deoxy-guanosine 108.

FIGS. 2A-B illustrate the hydrogen bonding between the purine and pyrimidine bases of two anti-parallel DNA strands.

FIG. 3 illustrates a short section of a DNA double helix 300 comprising a first strand 302 and a second, anti-parallel strand 304.

FIGS. 4-7 illustrate the principle of the array-based hybridization assay.

FIGS. 8A-B illustrate one class of feature-location anomalies that is observed in manufactured microarrays.

FIGS. 9A-B illustrate a first step undertaken in various embodiments of the present invention.

FIGS. 10A-B illustrate the partitioning of the initial region, illustrated in FIG. 9A, and calculation of the vector sums of the displacement vectors, μᵥ, the length of the vector sum, \overline{μ}ᵥ, and the average displacement-vector lengths, \overline{μ}ₛ, for each of the subpartitions.

FIGS. 11A-B illustrate that the partitions from FIG. 10A that are determined to include block misalignments, partitions 1004 and 1006, may be further subdivided into partitions.

FIG. 12 indicates that the subpartitions shown in FIG. 11A may be further partitioned, in order to more effectively isolate the block misalignments.

FIG. 13 is a flow-control diagram of the routine “feature positions” that represents one embodiment of the present invention.

FIG. 14 is a flow-control diagram for the routine “partition,” called in step 1310 of FIG. 13.

FIG. 15 is a flow-control diagram for the routine “add subpartitions” called in step 1418 in FIG. 14.

FIG. 16 is a flow-control diagram for the routine “coalesce,” called in step 1312 of FIG. 13.

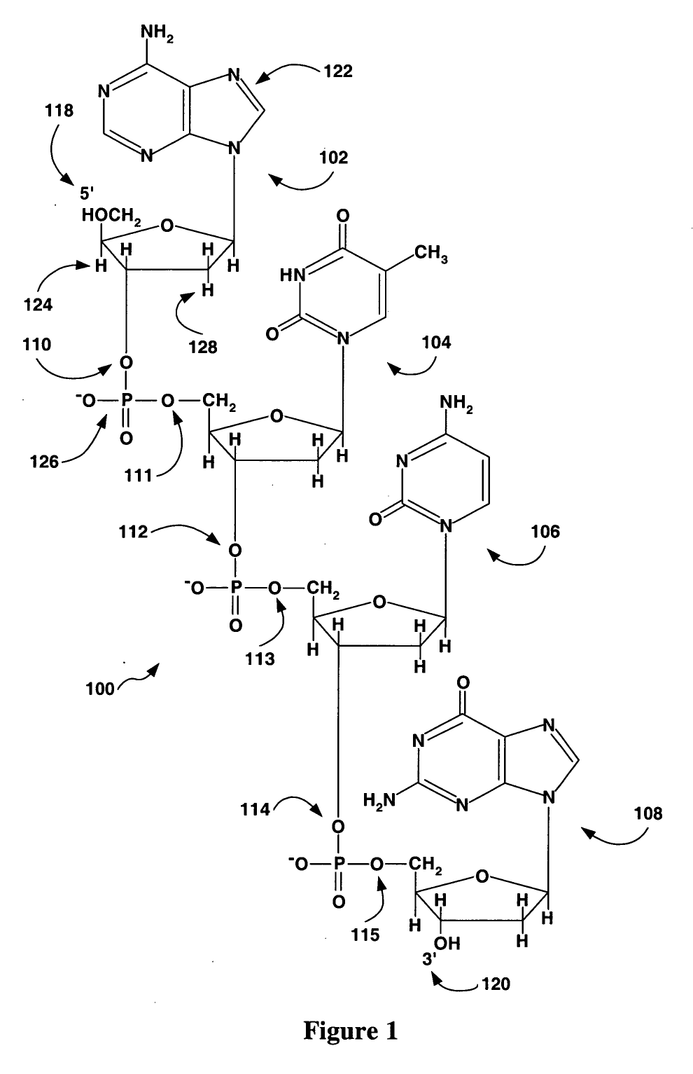

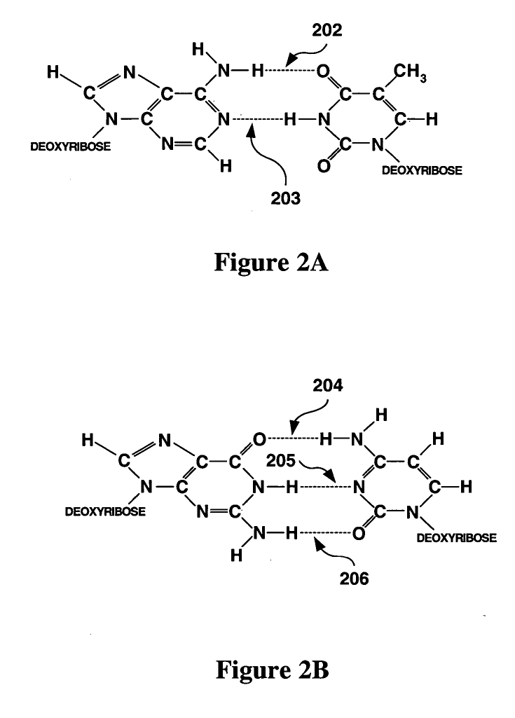

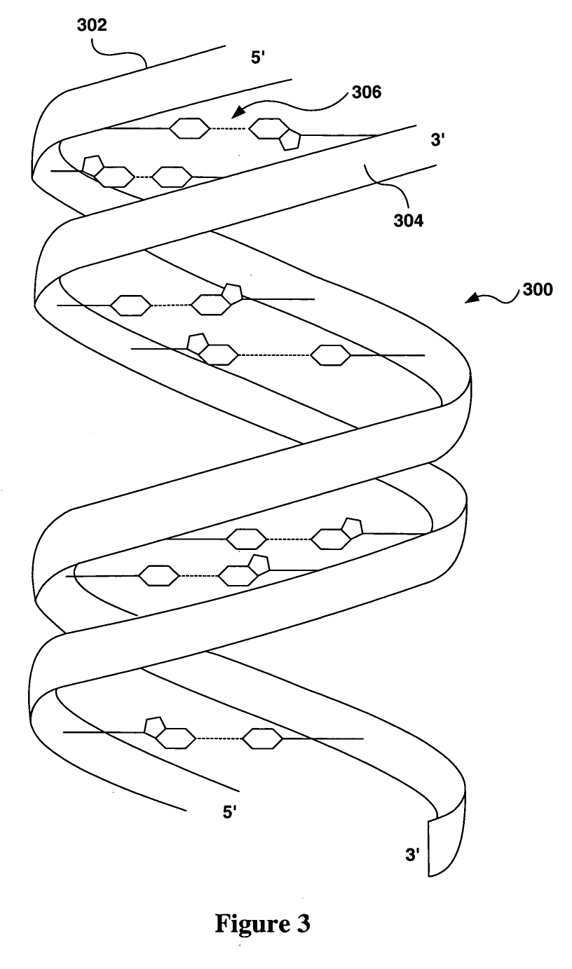

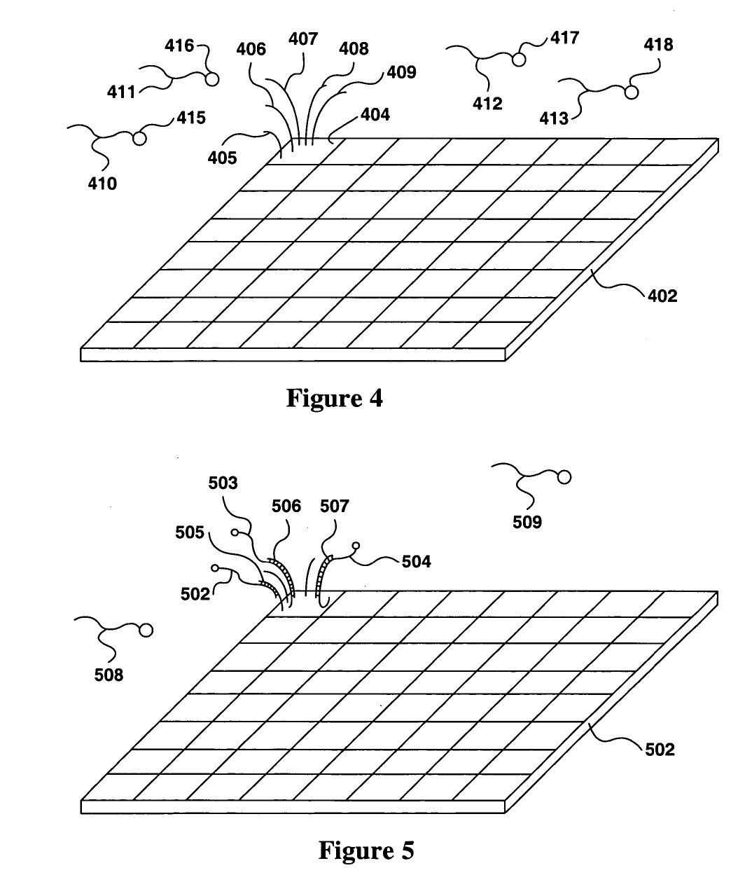

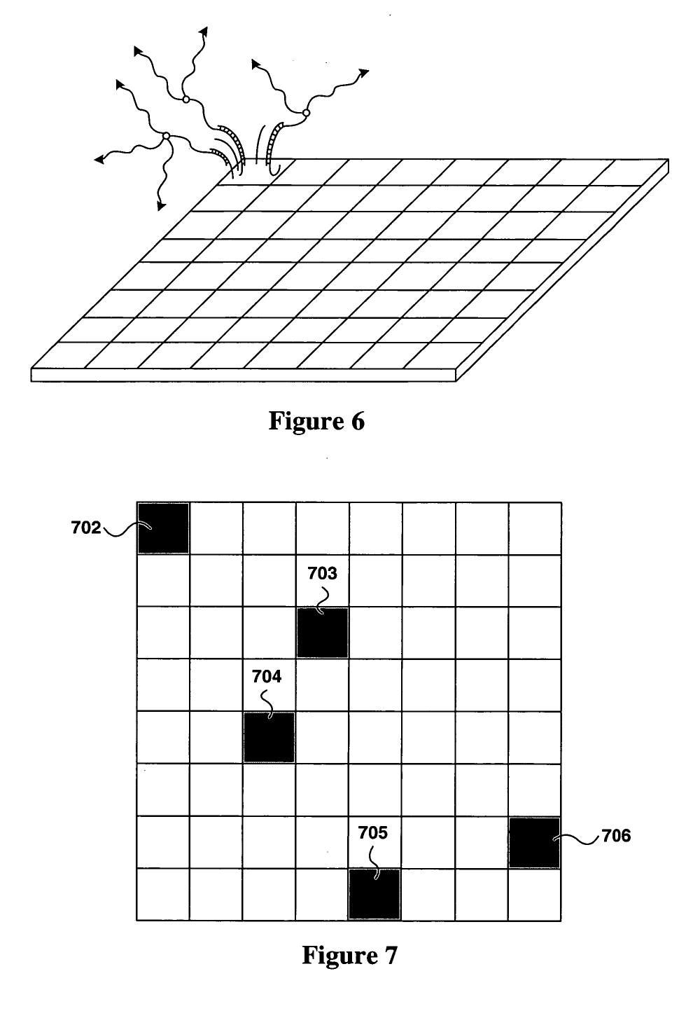

### DETAILED DESCRIPTION OF THE INVENTION

One embodiment of the present invention provides a method and system for detecting and correcting for block and zone misalignments within a microarray data set. In a first subsection, below, additional information about molecular arrays is provided. Those readers familiar with molecular arrays may skip over this first subsection. In a second subsection, embodiments of the present invention are provided through examples, graphical representations, and with reference to several flow-control diagrams.

### Additional Information About Molecular Arrays

An array may include any one-, two- or three-dimensional arrangement of addressable regions, or features, each bearing a particular chemical moiety or moieties, such as biopolymers, associated with that region. Any given array substrate may carry one, two, or four or more arrays disposed on a front surface of the substrate. Depending upon the use, any or all of the arrays may be the same or different from one another and each may contain multiple spots or features. A typical array may contain more than ten, more than one hundred, more than one thousand, more ten thousand features, or even more than one hundred thousand features, in an area of less than 20 cm² or even less than 10 cm². For example, square features may have widths, or round feature may have diameters, in the range from a 10 μm to 1.0 cm. In other embodiments each feature may have a width or diameter in the range of 1.0 μm to 1.0 mm, usually 5.0 μm to 500 μm, and more usually 10 μm to 200 μm. Features other than round or square may have area ranges equivalent to that of circular features with the foregoing diameter ranges. At least some, or all, of the features may be of different compositions (for example, when any repeats of each feature composition are excluded the remaining features may account for at least 5%, 10%, or 20% of the total number of features). Inter-feature areas are typically, but not necessarily, present. Inter-feature areas generally do not carry probe molecules. Such inter-feature areas typically are present where the arrays are formed by processes involving drop deposition of reagents, but may not be present when, for example, photolithographic array fabrication processes are used. When present, inter-feature areas can be of various sizes and configurations.

Each array may cover an area of less than 100 cm², or even less than 50 cm², 10 cm² or 1 cm². In many embodiments. the substrate carrying the one or more arrays will be shaped generally as a rectangular solid having a length of more than 4 mm and less than 1 m, usually more than 4 mm and less than 600 mm, more usually less than 400 mm; a width of more than 4 mm and less than 1 m, usually less than 500 mm and more usually less than 400 mm; and a thickness of more than 0.01 mm and less than 5.0 mm, usually more than 0.1 mm and less than 2 mm and more usually more than 0.2 and less than 1 mm. Other shapes are possible, as well. With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, a substrate may transmit at least 20%, or 50% (or even at least 70%, 90%, or 95%), of the illuminating light incident on the front as may be measured across the entire integrated spectrum of such illuminating light or alternatively at 532 nm or 633 nm.

Arrays can be fabricated using drop deposition from pulsejets of either polynucleotide precursor units (such as monomers) in the case of in situ fabrication, or the previously obtained polynucleotide. Such methods are described in detail in, for example, U.S. Pat. Nos. 6,242,266, 6,232,072, 6,180,351, 6,171,797, 6,323,043, U.S. patent application Ser. No. 09/302,898 filed Apr. 30, 1999 by Caren et al., and the references cited therein. Other drop deposition methods can be used for fabrication, as previously described herein. Also, instead of drop deposition methods, photolithographic array fabrication methods may be used. Inter-feature areas need not be present particularly when the arrays are made by photolithographic methods as described in those patents.

A microarray is typically exposed to a sample including labeled target molecules, or, as mentioned above, to a sample including unlabeled target molecules followed by exposure to labeled molecules that bind to unlabeled target molecules bound to the array, and the array is then read. Reading of the array may be accomplished by illuminating the array and reading the location and intensity of resulting fluorescence at multiple regions on each feature of the array. For example, a scanner may be used for this purpose, which is similar to the AGILENT MICROARRAY SCANNER manufactured by Agilent Technologies, Palo Alto, Calif. Other suitable apparatus and methods are described in U.S. patent application Ser. No. 10/087447 “Reading Dry Chemical Arrays Through The Substrate” by Corson et al., and in U.S. Pat. Nos. 6,518,556; 6,486,457; 6,406,849; 6,371,370; 6,355,921; 6,320,196; 6,251,685; and 6,222,664. However, arrays may be read by any other method or apparatus than the foregoing, with other reading methods including other optical techniques, such as detecting chemiluminescent or electroluminescent labels, or electrical techniques, for where each feature is provided with an electrode to detect hybridization at that feature in a manner disclosed in U.S. Pat. Nos. 6,251,685, 6,221,583 and elsewhere.

A result obtained from reading an array may be used in that form or may be further processed to generate a result such as that obtained by forming conclusions based on the pattern read from the array, such as whether or not a particular target sequence may have been present in the sample, or whether or not a pattern indicates a particular condition of an organism from which the sample came. A result of the reading, whether further processed or not, may be forwarded, such as by communication, to a remote location if desired, and received there for further use, such as for further processing. When one item is indicated as being remote from another, this is referenced that the two items are at least in different buildings, and may be at least one mile, ten miles, or at least one hundred miles apart. Communicating information references transmitting the data representing that information as electrical signals over a suitable communication channel, for example, over a private or public network. Forwarding an item refers to any means of getting the item from one location to the next, whether by physically transporting that item or, in the case of data, physically transporting a medium carrying the data or communicating the data.

As pointed out above, array-based assays can involve other types of biopolymers, synthetic polymers, and other types of chemical entities. A biopolymer is a polymer of one or more types of repeating units. Biopolymers are typically found in biological systems and particularly include polysaccharides, peptides. and polynucleotides, as well as their analogs such as those compounds composed of, or containing, amino acid analogs or non-amino-acid groups, or nucleotide analogs or non-nucleotide groups. This includes polynucleotides in which the conventional backbone has been replaced with a non-naturally occurring or synthetic backbone, and nucleic acids, or synthetic or naturally occurring nucleic-acid analogs, in which one or more of the conventional bases has been replaced with a natural or synthetic group capable of participating in Watson-Crick-type hydrogen bonding interactions. Polynucleotides include single or multiple-stranded configurations, where one or more of the strands may or may not be completely aligned with another. For example, a biopolymer includes DNA, RNA, oligonucleotides, and PNA and other polynucleotides as described in U.S. Pat. No. 5,948,902 and references cited therein, regardless of the source. An oligonucleotide is a nucleotide multimer of about 10 to 100 nucleotides in length, while a polynucleotide includes a nucleotide multimer having any number of nucleotides.

As an example of a non-nucleic-acid-based microarray, protein antibodies may be attached to features of the array that would bind to soluble labeled antigens in a sample solution. Many other types of chemical assays may be facilitated by array technologies. For example, polysaccharides, glycoproteins, synthetic copolymers, including block copolymers, biopolymer-like polymers with synthetic or derivitized monomers or monomer linkages, and many other types of chemical or biochemical entities may serve as probe and target molecules for array-based analysis. A fundamental principle upon which arrays are based is that of specific recognition, by probe molecules affixed to the array, of target molecules, whether by sequence-mediated binding affinities, binding affinities based on conformational or topological properties of probe and target molecules, or binding affinities based on spatial distribution of electrical charge on the surfaces of target and probe molecules.

Scanning of a microarray by an optical scanning device or radiometric scanning device generally produces a scanned image comprising a rectilinear grid of pixels, with each pixel having a corresponding signal intensity. These signal intensities are processed by an array-data-processing program that analyzes data scanned from an array to produce experimental or diagnostic results which are stored in a computer-readable medium, transferred to an intercommunicating entity via electronic signals, printed in a human-readable format, or otherwise made available for further use. Molecular array experiments can indicate precise gene-expression responses of organisms to drugs, other chemical and biological substances, environmental factors, and other effects. Molecular array experiments can also be used to diagnose disease, for gene sequencing, and for analytical chemistry. Processing of microarray data can produce detailed chemical and biological analyses, disease diagnoses, and other information that can be stored in a computer-readable medium, transferred to an intercommunicating entity via electronic signals, printed in a human-readable format, or otherwise made available for further use.

### Embodiments of the Present Invention

One embodiment of the present invention provides a method and system for detecting block and zone misalignments of feature positions within a microarray-data set and for correcting feature positions for block or zone misalignment. A block or zone can be a physically contiguous set of features or can be a set of features defined by print-tip or nozzle membership, such as, for example, a set of features printed from the same print-tip or nozzle among other printing devices. Block and zone misalignments are sets of features translated, rotated, or rotated and translated with respect to initially expected positions for the features based on an initially determined coordinate grid derived from the determined positions of a subset of features. The terms “block misalignment” and “zone misalignment” are essentially interchangeable, although the term “block misalignment” may be more appropriate for relatively smaller regions of misalignment, while the term “zone misalignment” may be more appropriate for relatively larger regions of misalignment. The term “block/zone misalignment” is used, below, to refer to either a block or zone misalignment. In one embodiment of the present invention, displacement vectors representing the vector differences between observed positions of features and expected positions for the features of a microarray are calculated, based on an initially determined coordinate system. Features within a microarray data set are then partitioned with respect to the calculated vector displacements, so that features misaligned by a common rotation or translation are partitioned into a separate partition. A correction for the common misalignment of the features of the block or zone can then be calculated and applied to the features of the block or zone.

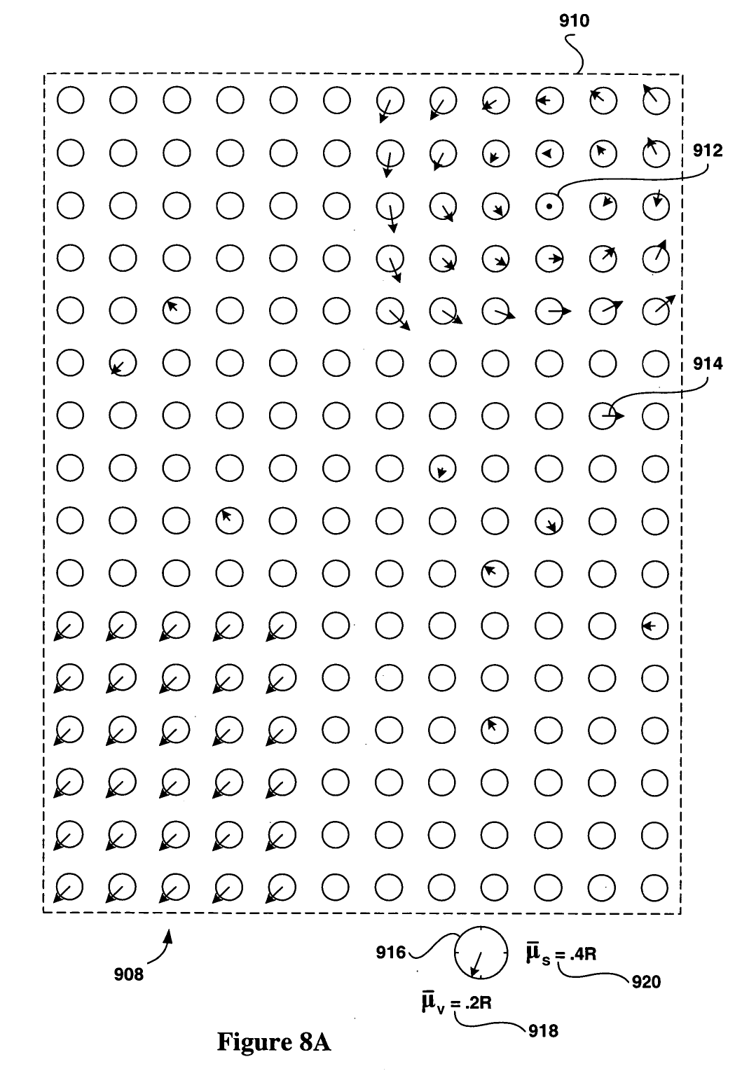

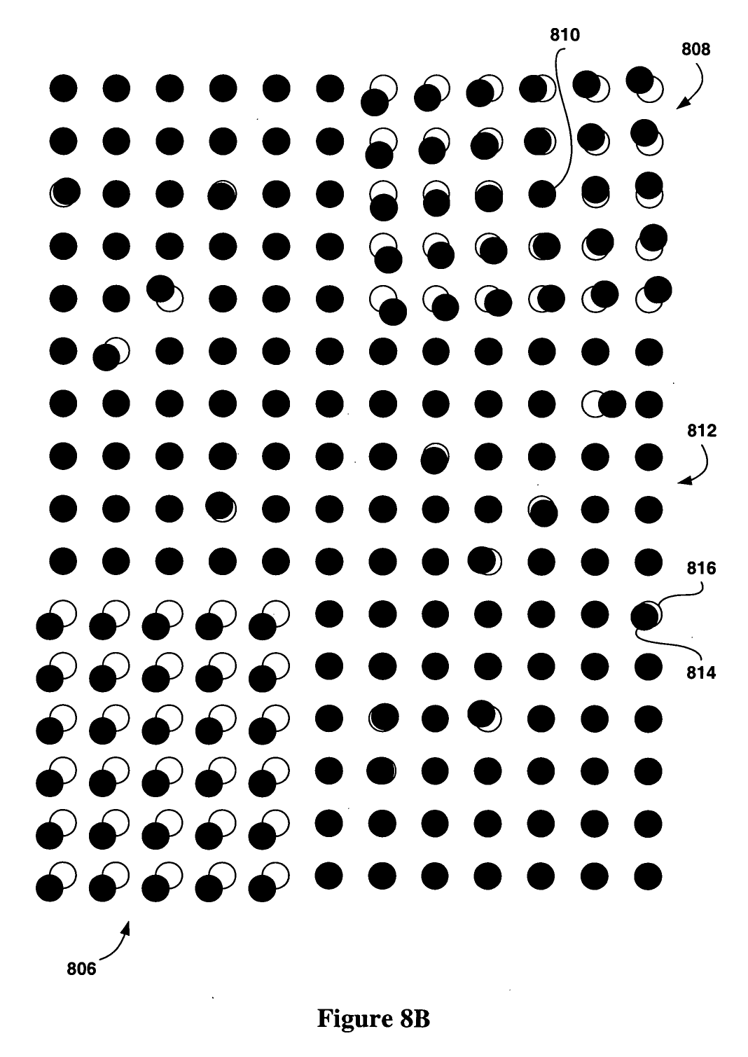

FIGS. 8A-B illustrate one class of feature-location anomalies that is observed in manufactured microarrays. As shown in FIG. 8A, the desired locations for features, in one class of microarrays, are rectilinear, perfectly periodic feature positions in a two-dimensional space. This rectilinear, grid-like pattern of feature positions, such as feature position 801, represented in FIG. 8A by an unfilled disk, can be characterized by a horizontal spacing 802 between features and a vertical spacing 804 between features. Were the features all perfectly positioned, then knowing the exact location of one, known feature, the location of any other feature could be found relative to the known feature by applying a number of horizontal and vertical translations. Unfortunately, as shown in FIG. 8B, microarray manufacturing processes may introduce various systematic feature-position errors, as well as pseudo-randomly or randomly distributed feature-position errors. In FIG. 8B, the observed feature locations are indicated by filled disks, while the expected feature locations are indicated by unfilled disks, as in FIG. 8A. In FIG. 8B, a five-by-six subregion of features 806 is translated diagonally downward and leftward, as a group, thus representing a block diagonal translocation. A second, six-by-five subregion of features 808 is rotated counterclockwise about feature 810, the subregion therefore representing a block misalignment due to rotation. In the remaining portion of the two-dimensional feature lattice 812, various pseudo-random or random, individual-feature-position errors are observed, such as the leftward and downward translocation of feature 814 with respect to its expected position 816.

As discussed above, each feature can be separately analyzed in order to reconcile the observed location, or location calculated from observed locations of nearby known features, with the location expected from the most recently calculated coordinate axes. However, a feature-by-feature repositioning approach may fail to take into account systematic error information for misaligned feature blocks and feature zones that can potentially provide greater accuracy for feature location, particularly in the case of features with low signal-to-noise ratios.

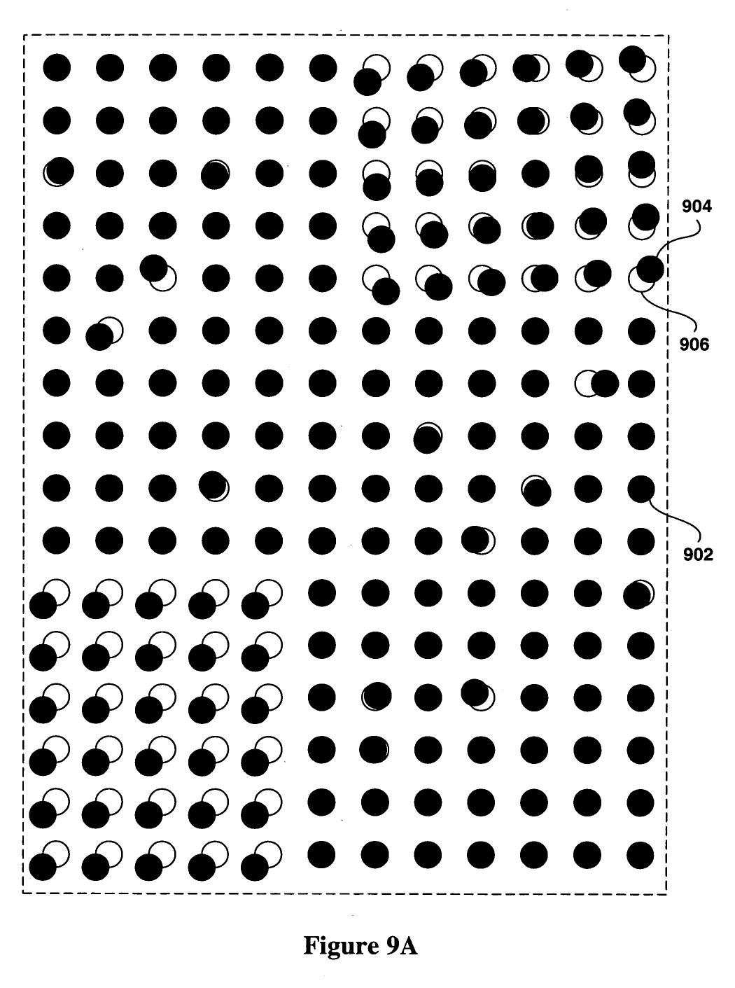

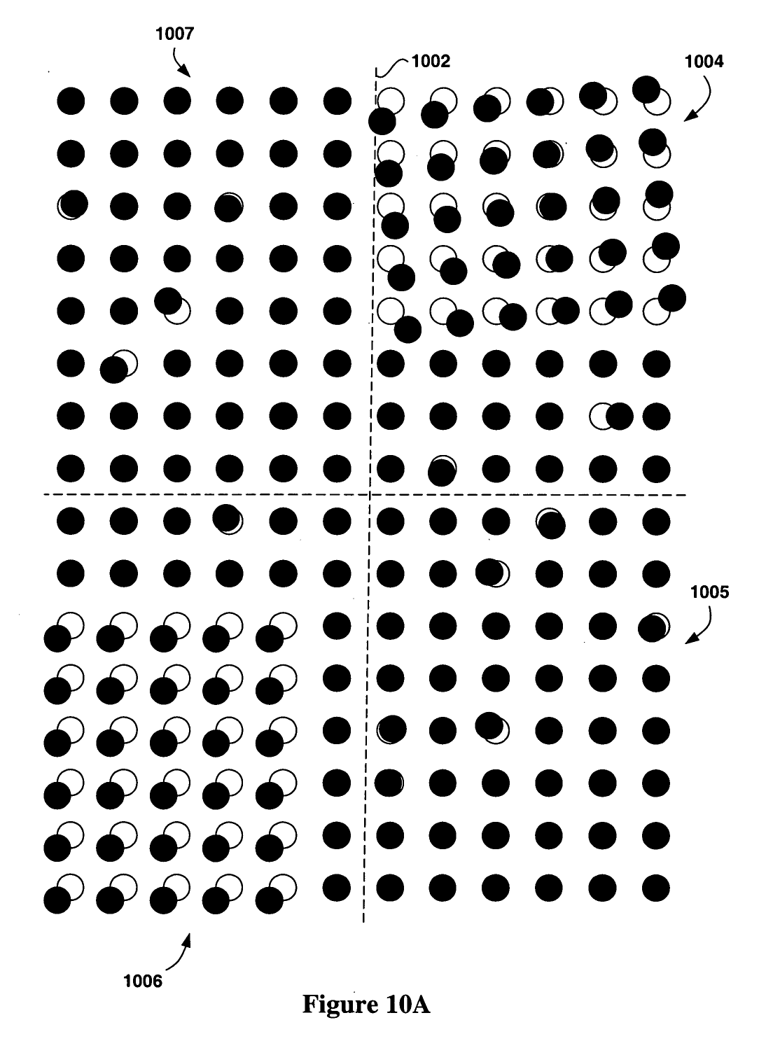

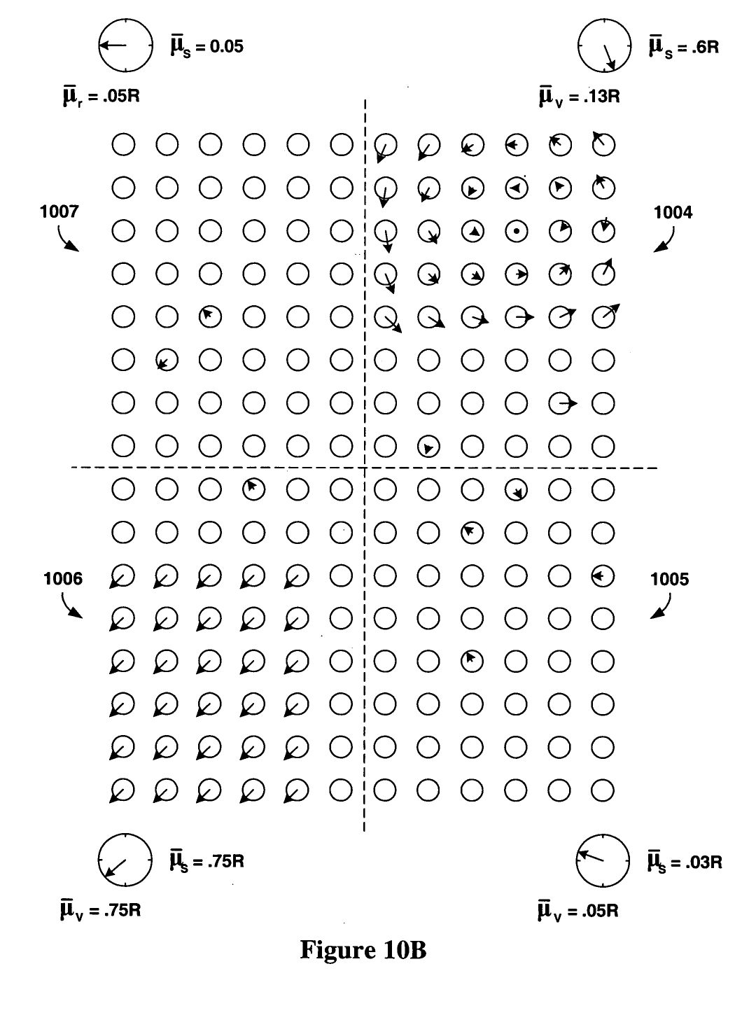

FIGS. 9A-B illustrate a first step undertaken in various embodiments of the present invention. FIG. 9 illustrates an initial microarray data set containing several block misalignments as well as various random feature-position errors. In FIG. 9A, as in FIGS. 10A, 11A, and 12 referenced below, filled disks represent observed feature positions and unfilled disks represent expected feature positions. For most features, the observed position is coincident with the expected feature position, resulting in a single filled disk, such as for feature 902. However, for some features, the observed feature position is offset from the expected feature position, as, for example, the observed feature position 904 is offset from the expected feature position 906. Of course, an actual microarray data set may contain thousands of, many tens of thousands of, or more features, and the small data set illustrated in FIG. 9A is employed for convenience and clarity in illustration only. In a first step to identifying block misalignments, as shown in FIG. 9B, displacement vectors are calculated for each feature. In FIG. 9B, for example, a block of features in the lower, left-hand corner are translated from their expected positions in a leftward and downward diagonal direction. Thus, the displacement vectors for each of these features has a direction coincident with the downward and leftward translation. A displacement vector may be calculated by subtracting the expected feature centroid position from the observed feature centroid position. Similarly, a block of features in the upper right-hand corner 910 exhibits a rotational misalignment, where the rotation can be easily observed by the counter clockwise, circular pattern of the vector directions about the feature 912. A number of pseudo-randomly oriented displacement vectors, such as displacement vector 914, can be seen in the remaining portion of the microarray feature positions.

Of course, displacement vectors may be oriented from the expected feature position to the observed feature position, or may be oriented in an exactly opposite direction from the observed feature position to the expected feature position, depending on the order of positions in the subtraction operation used to generate a displacement vector. Displacement vectors may be appropriately scaled for visual display and displayed superimposed over feature positions in a displayed image of the feature positions, in order to assist an experimenter in visually identifying block misalignments.

Several different cumulative displacement-vector-based metrics can be calculated from the displacement vectors for the partition. A first metric is the vector sum of the displacement vectors for a region within a microarray, μᵥ, calculated by summing all displacement vectors dᵢ in the region, as follows:

$${μ}_{v} = {\sum }_{i = 1}^{n} {d}_{i}$$

A second metric that can be calculated is the length, or magnitude, of the vector sum of the displacement vectors, \overline{μ}ᵥ, calculated as:
\overline{μ}ᵥ=\sqrt{μᵥ·μᵥ}
Finally, a third metric that can be calculated is the average length of the displacement vectors within the region, \overline{μ}ₛ, calculated as follows:

$${\overline{μ}}_{s} = \frac{1}{n} {\sum }_{i = 1}^{n} \sqrt{{d}_{i} \cdot {d}_{i}}$$

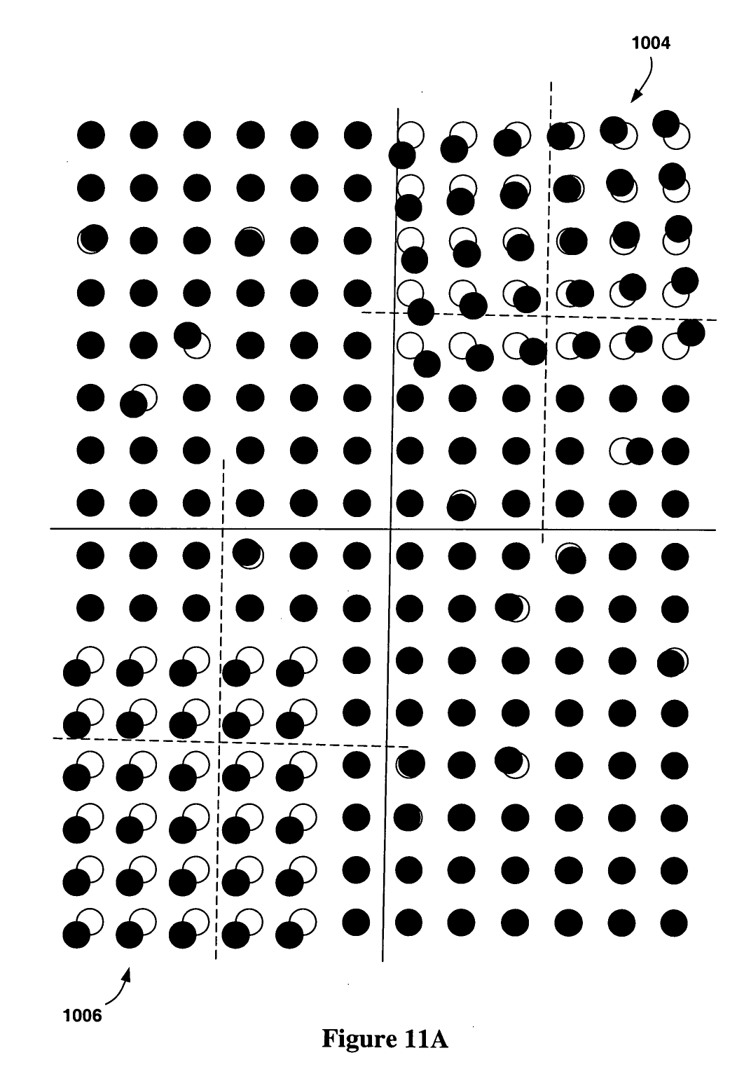

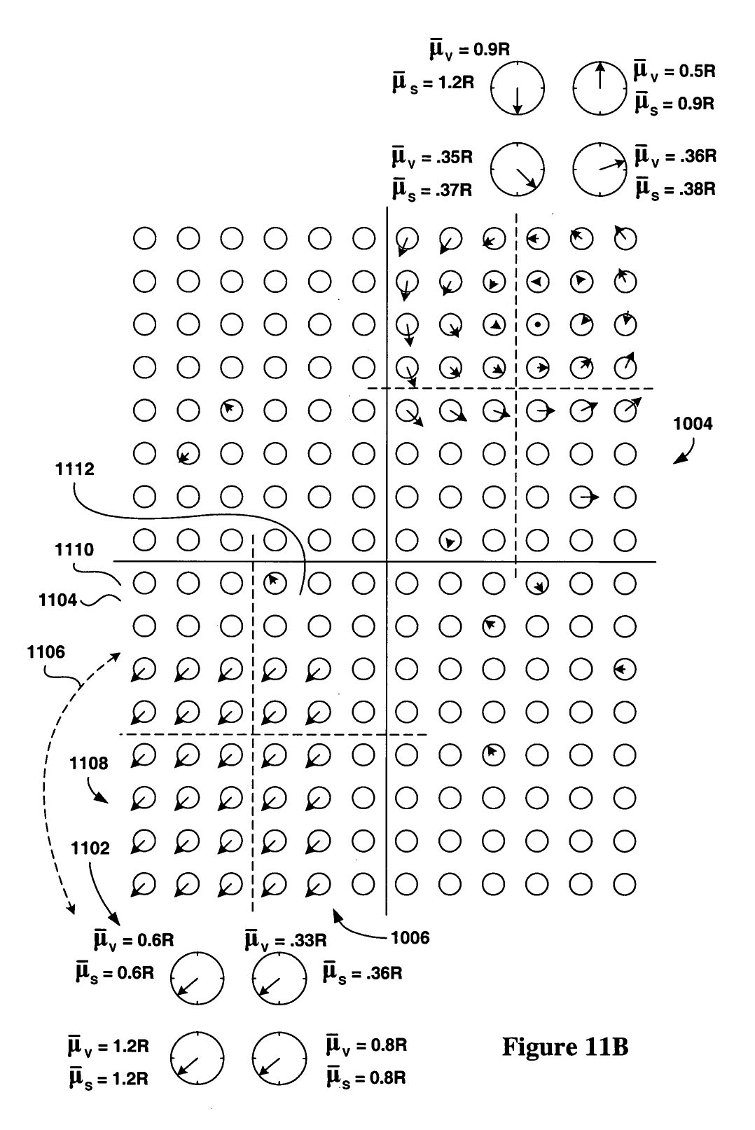

In FIG. 9B, and in the following FIGS. 10B and 11B, the direction of the vector sum μᵥ is shown in a compass-type FIG. 916, and the scaler values for the length of the vector sum \overline{μ}ᵥ and the sum of the length of the displacement vectors \overline{μ}ₛ 918 and 920 are provided in units of R, where R is the median radius of all the feature disks in the region. Expressing the lengths in units of R is employed for convenience in describing the figures, since the median feature-disk radius is the unit most available in the example illustrations. This unit convention would probably not be chosen in an implementation of the described methods, because the boundary of feature disks may not be circular, and thus may not have easily defined radii. Also, although the median feature radius would probably vary with various partitions, it is assumed to be a constant, in the current examples.

Once the vector sum of the displacement vectors, μᵥ, the length of the vector sum, \overline{μ}ᵥ, and the sum of the length of the displacement vectors, \overline{μ}ₛ are computed for the overall region, shown in FIG. 9B, the region may be subdivided, or partitioned, into subregions or subpartitions. There are a variety of ways to partition a region, some more appropriate than others, depending on the geometry and spacing of features on the surface of the microarray. For rectilinear grid-like feature positions, subdividing an initial partition into four, equally-sized rectangular partitions is an easily computed and convenient partitioning approach. In alternate embodiments, when the identity of an individual feature-depositing pin or nozzle is known, the partitioning may be undertaken to partition the features into sets of features deposited by the same, or a mechanically related, pin or nozzle. FIGS. 10A-B illustrate the partitioning of the initial region, illustrated in FIG. 9A, and calculation of the vector sums of the displacement vectors, μᵥ, the length of the vector sum, \overline{μ}ᵥ, and the average displacement-vector lengths, \overline{μ}ₛ, for each of the subpartitions. In FIG. 10A, dashed lines, such as dashed line 1002, are used to horizontally and vertically bisect the initial region, creating four subpartitions 1004-1007. FIG. 10B shows the directions and lengths of the vector sums of the displacement vectors for each partition, as well as the scaler value representing the average displacement-vector lengths in each partition, \overline{μ}ₛ. As can be seen in FIG. 10B, in comparing the calculated vector-sum direction, length, and average displacement-vector length for each partition to those calculated values for the initial region, shown in FIG. 9B, the calculated vector-sum direction and length and average displacement vector length markedly varies over the initial region, shown in FIG. 9B. For example, for partition 1004, the length of the vector sum of the displacement vectors 0.13 R is substantially less than that for the entire region, 0.2 R, while the average displacement-vector length 0.6 R is substantially greater than the average vector-displacement length 0.4 R for the entire region.

When the ratio

$$\frac{{\overline{μ}}_{v}}{{\overline{μ}}_{s}}$$

falls significantly below 1.0, there is a strong indication of a rotational misalignment within the partition, since the vector sum of displacement vectors about a rotation point, such as rotation point 912, are symmetrical and tend to cancel each other out. The fact that the average displacement-vector length \overline{μ}ₛ is greater in partition 1004 than in the original region indicates that the partitioning has potentially isolated a block misalignment within the partition, since the features within the partition have a greater, average displacement-vector length than the features of the initial region, in general. Similarly, for partition 1006, the increase in the average displacement-vector length from 0.4 R to 0.75 R indicates the presence, within the partition, of a block misalignment, but the increase in the ratio

$$\frac{{\overline{μ}}_{v}}{{\overline{μ}}_{s}}$$

from 0.5 to 1.0 indicates that a rotational misalignment present in the initial region is no longer present, or present to a much smaller extent, in partition 1006. The fact that the average displacement-vector length for partitions 1005 and 1007 have markedly decreased indicates that these partitions do not include block misalignments.

FIGS. 11A-B illustrate that the partitions from FIG. 10A that are determined to include block misalignments, partitions 1004 and 1006, may be further subdivided into partitions. FIG. 11B shows the direction and length of the vector sums of the displacement vectors and the average length of the vector displacements for each of the subpartitions shown in FIG. 11A. The vector-sum directions and lengths are shown in spatial correspondence with, but diagonally offset from, the partitions in FIG. 11B. Thus, for example, the vector-sum directions and lengths 1102 correspond to subpartition 1104, as indicated by the dashed arrow 1106.

With regard to partition 1004, the increase in the ratio

$$\frac{{\overline{μ}}_{v}}{{\overline{μ}}_{s}}$$

for each of the subpartitions within partition 1004 indicates that the subpartitions have partitioned a block rotational misalignment, and thus the partition 1004 is probably best not further partitioned in order that partition 1004 fully includes the block rotational misalignment. With regard to the partition 1006, the fact that the average vector-displacement length \overline{μ}ₛ markedly increases for the lower-left subpartition 1108 indicates that subpartition 1108 has isolated a block misalignment more effectively than partition 1006, in which it is included. However, the fact that the average vector-displacement length \overline{μ}ₛ has decreased for the upper two subpartitions 1110 and 1112 indicates that the ratio of features exhibiting a block misalignment within those subpartitions to all features within the subpartitions is less than the ratio for the larger including partition 1006. Thus, a repartitioning of partition 1006, or repartitioning of all but subpartition 1108, may be needed in order to isolate the block translational misalignment.

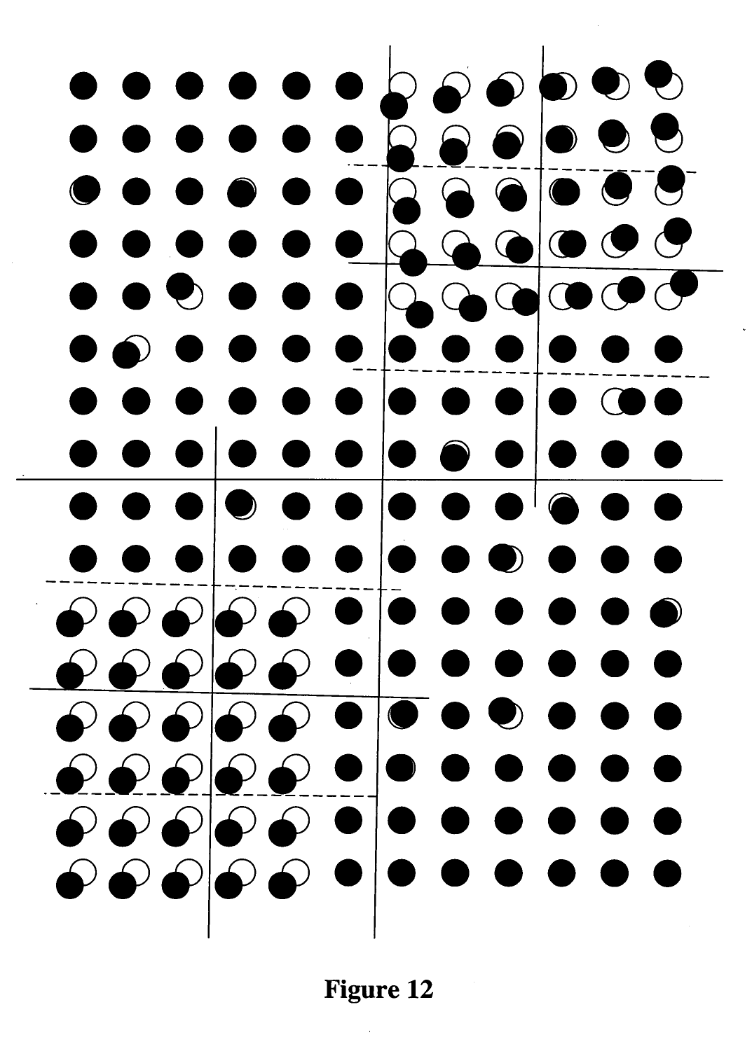

FIG. 12 indicates that the subpartitions shown in FIG. 11A may be further partitioned, in order to more effectively isolate the block misalignments. The process of partitioning may continue until either rotational misalignments are isolated or until the average length of displacement vectors within a subpartition falls below a threshold value, indicating that no block misalignment is present in the subpartition or until the average length of the displacement vectors in a subpartition is equal to that of the parent partition that includes a subpartition, indicating that further partitioning is unnecessary.

With the principles of microarray-region partitioning and vector-displacement-based metrics described with reference to FIG. 9-12, an embodiment of a block-and-zone-misalignment detection-and-correction method can now be provided. This embodiment is described with reference to four flow-control diagrams, below, as well as to a series of mathematical equations that follow.

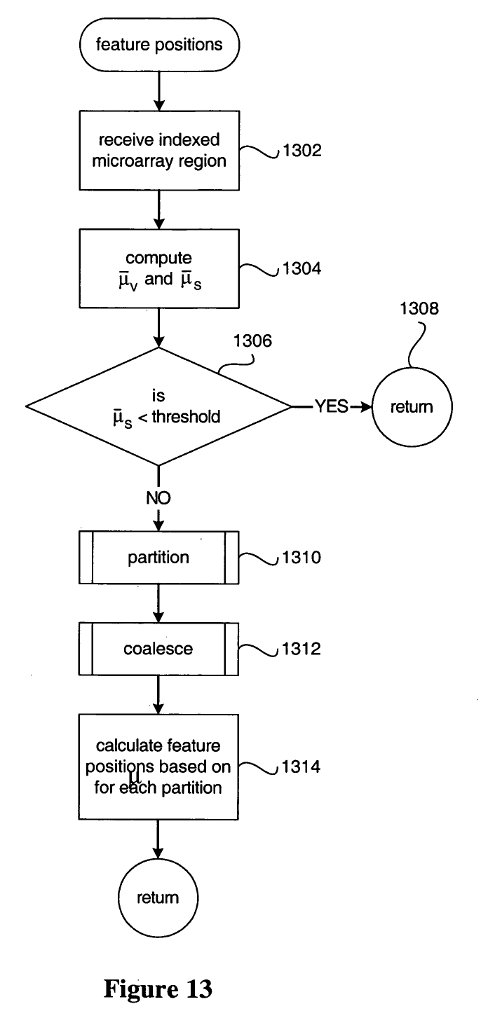

FIG. 13 is a flow-control diagram of the routine “feature positions” that represents one embodiment of the present invention. In step 1302, the routine “feature positions” receives an indexed microarray region obtained by an initial feature indexing method or, in other words, a microarray region along with an initially computed coordinate system by which the position of each feature can be estimated. In step 1304, the routine “feature positions” computes the vector sum of the vector displacements, μᵥ, the length of the vector sum, \overline{μ}ᵥ, and the average length of vector displacements \overline{μ}ₛ for all or a selected, well-distributed portion of the features of the region. In step 1306, the routine “feature positions” determines whether the average vector-displacement length \overline{μ}ₛ is less than a threshold value. If so, then no block misalignment is detectable within the region, and the routine “feature positions” returns in step 1308. Otherwise, the routine “feature positions” calls the recursive routine “partition,” in step 1310, in order to partition the region into subpartitions, each of which includes a block misalignment, and then calls the routine “coalesce,” in step 1312, to coalesce partitions, when possible, so that the largest partitions including particular block misalignments are obtained. Then, in step 1314, the routine “feature positions” recalculates the feature positions for each feature, taking into account any detected block misalignments. This can be done by applying calculated displacement vectors to estimated feature positions in a matrix operation. Calculation of feature positions and displacement vectors is discussed in detail, below. Step 1314 therefore represents the feature-position-correction step of the process that represents one embodiment of the present invention.

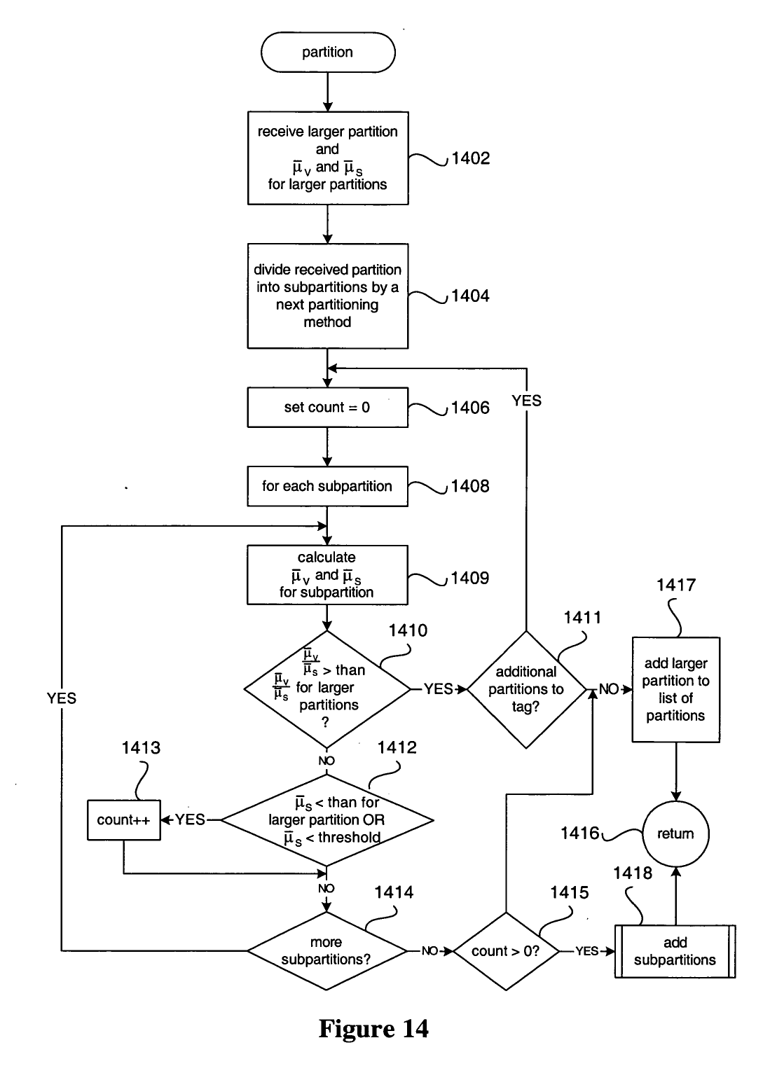

FIG. 14 is a flow-control diagram for the routine “partition,” called in step 1310 of FIG. 13. In step 1402, the routine “partition” receives a region or partition, referred to as the “parent partition,” for partitioning or re-partitioning into subpartitions. In addition, the routine “partition” receives the calculated vector-sum length of the displacement vectors and the average displacement-vector length, \overline{μ}ᵥ and \overline{μ}ₛ, for the parent partition. Next, in step 1404, the routine “partition” divides the received parent partition into subpartitions by a next partitioning method. The term “next partitioning method” is used to indicate that there may be a number of different partitioning methods may be attempted, when necessary, in order to arrive at the best possible partitioning for the parent partition. For example, if a partition cannot be evenly divided, then several different asymmetrical partitionings might be applied, in order to arrive at a best possible partitioning. As another example, geometric and other types of partitioning based of feature-deposition knowledge may both be tried. As discussed above, the type of partitioning methods available to the routine “partition” depends on the spacings and geometry of the feature-position locations on the surface of the microarray. In step 1406, the routine “partition” sets the local variable “count” to zero. Then, in a ƒor-loop comprising steps 1408-1415, each subpartition of the parent partition is evaluated. In step 1409, the length of the vector sum of the vector displacements, \overline{μ}ᵥ, and the average vector-displacement length, \overline{μ}ₛ, are calculated for the currently considered partition. Next, in step 1410, the ratio

$$\frac{{\overline{μ}}_{v}}{{\overline{μ}}_{s}}$$

for the currently considered partition is compared to that for the parent partition. If the ratio

$$\frac{{\overline{μ}}_{v}}{{\overline{μ}}_{s}}$$

for the currently considered partition is greater than that for the parent partition, then partitioning of a larger rotational misalignment is indicated, and control flows to step 1411 where the routine “partition” determines whether there are any additional partitioning methods to try. If so, then control flows back to step 1406. If not, then the parent partition is added to a list of partitions, and the routine “partition” returns, in step 1416. If the ratio

$$\frac{{\overline{μ}}_{v}}{{\overline{μ}}_{s}}$$

is less than or equal to the ratio for the parent partition, then, in step 1412, the routine “partition” determines whether the average displacement-vector length, \overline{μ}ₛ, for the currently considered subpartition is less than for the parent partition or whether the average vector displacement length, \overline{μ}ₛ, for the currently considered subpartition is less than a threshold value. If so, then control flows to step 1413, where the local variable “count” is incremented, indicating that currently considered subpartition is a candidate for further inclusion in a final list of partitions. Next, in step 1414, the routine “partition” determines whether there are more subpartitions to evaluate. If so, control flows back to step 1409. Otherwise, control flows to step 1415, in which the routine “partition” determines whether the local variable “count” is greater than zero. If so, then the routine “partition” calls the routine “add subpartitions” in step 1418.

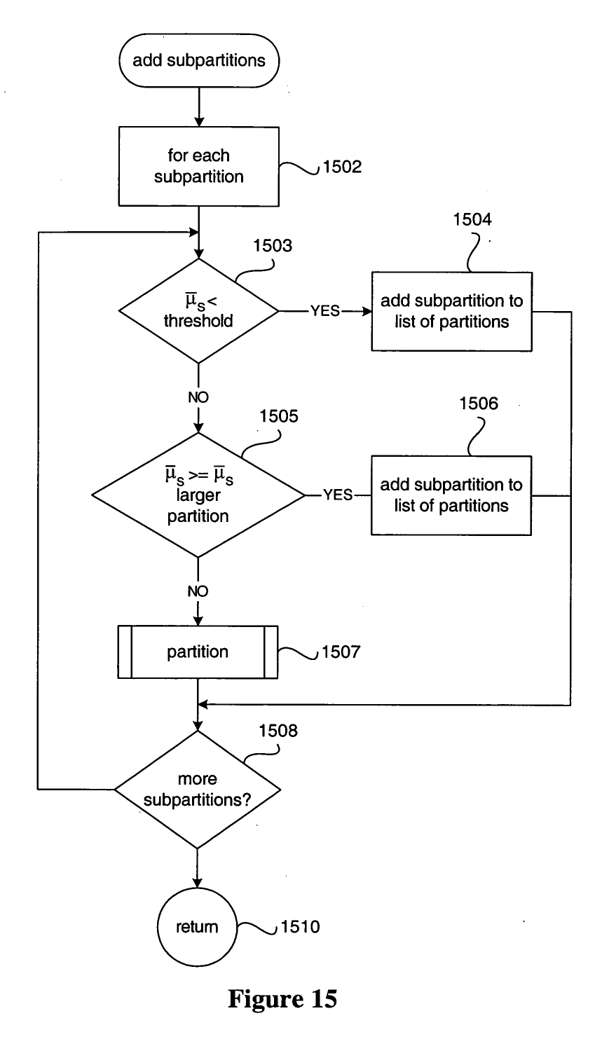

FIG. 15 is a flow-control diagram for the routine “add subpartitions” called in step 1418 in FIG. 14. Steps 1502-1508 together compose a ƒor-loop in which each subpartition of a partitioning carried out in step 1404 of FIG. 14 is evaluated. In step 1503, the routine “add subpartitions” determines whether the average vector-displacement length \overline{μ}ₛ is less than a threshold value. If so, then the subpartition is added to a list of partitions in step 1504. If not, then in step 1505, the routine “add subpartitions” determines whether the average vector-displacement length \overline{μ}ₛ is greater than or equal to the average vector-displacement length \overline{μ}ₛ for the parent partition. If so, then the subpartition is added to the list of partitions in step 1506. Otherwise, the routine “add subpartitions” recursively calls the routine “partition” in step 1507 to further partition the subpartition. If more subpartitions need to be considered, as detected in step 1508, then control flows back to step 1503. Otherwise, the routine “add subpartitions” returns to step 1510.

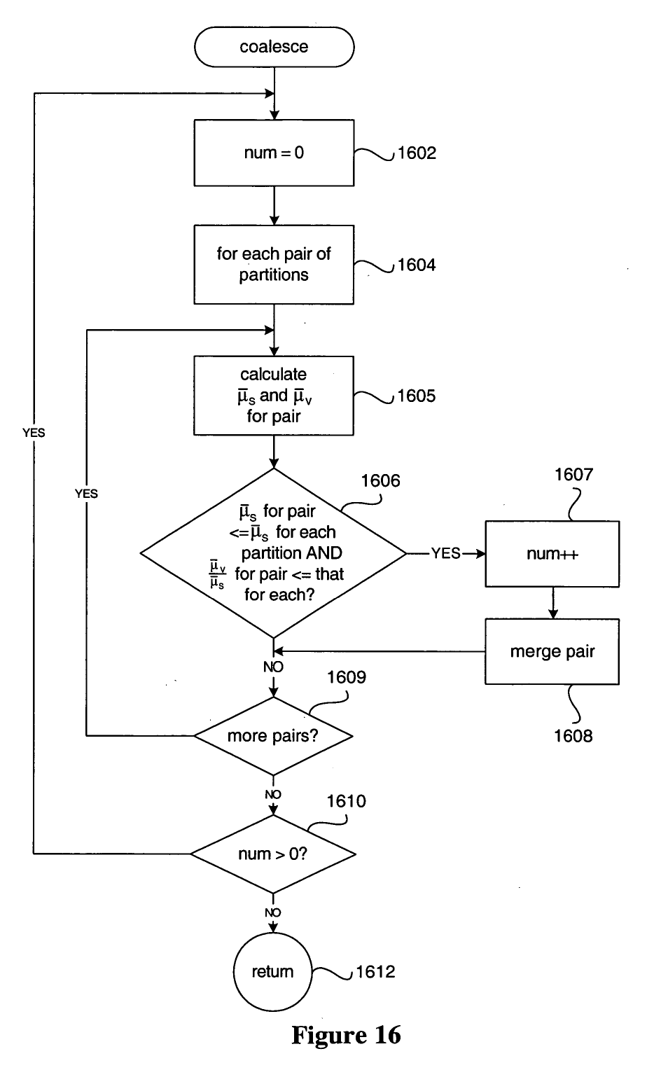

FIG. 16 is a flow-control diagram for the routine “coalesce,” called in step 1312 of FIG. 13. In step 1602, a local variable “num” is initialized to zero. Then, in the ƒor-loop comprising steps 1604-1610, coalesce attempts to merge pairs of partitions into larger partitions. In step 1605, the routine “coalesce” calculates the average vector-displacement length, \overline{μ}ₛ, and the length of the vector sum of the vector displacements, \overline{μ}ᵥ, for the combined pair of partitions. If the average vector-displacement length for the pair is less than or equal to that for each partition and the ratio

$$\frac{{\overline{μ}}_{v}}{{\overline{μ}}_{s}}$$

for the combined pair of partitions is less than or equal to the ratio for each partition, then, in step 1607, the routine “coalesce” increments the local variable “num” and, in step 1608, merges the currently considered pair of partitions together into a single partition. If there are more pairs of partitions to consider, as determined in step 1609, then control flows back to step 1605. Otherwise, if the local variable “num” is greater than zero, as determined in step 1610, then the routine “coalesce” repeats the ƒor-loop to attempt to additionally merge partitions. If no mergers occur in the current iteration, as detected in step 1610, the routine “coalesce” returns in step 1612. It should be emphasized that the coalescing step represented by the routine “coalesce” may, in many cases, prove to increase computational overhead more than the benefit obtained, and would be, in those cases, either omitted or used only in particular situations.

A method for calculating displacement vectors for the features in a partition is next provided. The location coordinates (xᵢ, yᵢ) for a feature can be calculated from the row and column index for the feature (rᵢ, cᵢ) as follows:
xᵢ=rᵢmₓₓ+cᵢm_{xy}+Oₓ
yᵢ=rᵢm_{yx}+cᵢm_{xy}+Oₓ
where
xᵢ is the horizontal position of the i^{th} feature,
yᵢ is the vertical position of the i^{th} feature,
rᵢ is the row of the i^{th} feature,
cᵢ is the column of the i^{th} feature,
mₓₓ is the horizontal projection of the horizontal spacing,
m_{yx} is the horizontal projection of the horizontal spacing,
m_{yy} is the horizontal projection of the horizontal spacing,
m_{xy} is the horizontal projection of the horizontal spacing,
Oₓ is the horizontal position of the grid origin, and
O_{y} is the vertical position of the grid origin.
This computation can be summarized using matrix algebra by incorporating the constants mₓₓ, m_{yy}, m_{yx}, m_{xy}, Oₓ, and O_{y} in a matrix M:

$$M = ( \begin{matrix}{m}_{xx} & {m}_{xy} & {O}_{x} \\ {m}_{yx} & {m}_{yy} & {O}_{y}\end{matrix} )$$

The matrix M can be computed from a matrix containing the centroids of found features C and the inverse of an index matrix I as follows:

$$M = {CI}^{- 1} where C = ( \begin{matrix}{x}_{1} & {y}_{1} \\ {x}_{2} & {y}_{2} \\ … & … \\ {x}_{n} & {y}_{n}\end{matrix} ) and I = ( \begin{matrix}1 & 1 & 1 & … & 1 \\ {c}_{1} & {c}_{2} & {c}_{3} & … & {c}_{n} \\ {r}_{1} & {r}_{2} & {r}_{3} & … & {r}_{n}\end{matrix} ) .$$

By matrix algebra, a matrix F including all the recalculated positions of a set of known features based on a block misalignment encapsulated in a matrix M can be determined as follows:
F_{found}=MI_{found}
More importantly, an estimate for the observed positions of all features within a partition, based on the matrix M determined using the observed positions of a set of known features, can be determined as follows:
Fₐₗₗ=MIₐₗₗ
A matrix of D displacement vectors can be then calculated for the features of a partition using the following expression:
D=C−F_{found}

Although the present invention has been described in terms of a particular embodiment, it is not intended that the invention be limited to this embodiment. Modifications within the spirit of the invention will be apparent to those skilled in the art. For example, many different partitioning methods may be employed in step 1404 of FIG. 14 in order to partition a given region of a microarray into subpartitions. Although the metrics \overline{μ}ᵥ, μᵥ, and \overline{μ}ₛ are used in the above-described embodiment, other vector-displacement-metrics may be used instead. And almost limitless number of different embodiments are possible, depending on in what medium the method is implemented and on details of implementation. For example, embodiments may be implemented in hardware, software, firmware, or a combination of two or more of hardware, software, and firmware, and software or logic may have many different modular organizations, use any of different control and data structures, and, in the case of software implementations, may be written in any of numerous different programming languages.

The foregoing description, for purposes of explanation, used specific nomenclature to provide a thorough understanding of the invention. However, it will be apparent to one skilled in the art that the specific details are not required in order to practice the invention. The foregoing descriptions of specific embodiments of the present invention are presented for purpose of illustration and description. They are not intended to be exhaustive or to limit the invention to the precise forms disclosed. Obviously many modifications and variations are possible in view of the above teachings. The embodiments are shown and described in order to best explain the principles of the invention and its practical applications, to thereby enable others skilled in the art to best utilize the invention and various embodiments with various modifications as are suited to the particular use contemplated. It is intended that the scope of the invention be defined by the following claims and their equivalents:

1. A method for identifying block/zone misalignments in a microarray data set, the method comprising:
calculating displacement vectors and displacement-vector-based metrics for the microarray data set comprising a digital image of a microarray and an initial feature indexing; and
partitioning the microarray data set into smaller partitions until each block/zone misalignment is isolated in one or more partitions, as determined by displacement-vector-based metrics calculated for each partition.

2. The method of claim 1 wherein displacement-vector-based metrics include:
a vector sum of the displacement vectors for a partition within a microarray calculated by summing all displacement vectors dᵢ in the partition according to
$${μ}_{v} = {\sum }_{i = 1}^{n} {d}_{i}$$
where μ, is the a vector sum of the displacement vectors and n is the number of features;
a length of the vector sum of the displacement vectors within the partition calculated according to
\overline{μ}ᵥ=\sqrt{μᵥ·μᵥ}
where \overline{μ}ᵥ is the length of the vector sum of the displacement vectors; and
an average length of the displacement vectors dᵢ within the partition, calculated according to
$${\overline{μ}}_{s} = \frac{1}{n} {\sum }_{i = 1}^{n} \sqrt{{d}_{i} \cdot {d}_{i}}$$
where \overline{μ}ₛ is the average length of the displacement vectors.

3. The method of claim 2 wherein partitioning the microarray data set into smaller partitions until block/zone misalignments are each isolated in one or more partitions further includes:
partitioning the microarray data into partitions until one of
\overline{μ}ₛ calculated for a partition falls below a threshold value,
\overline{μ}ₛ calculated for a final partition is less than or equal to \overline{μ}ₛ calculated for a larger partition that contains the final partition, or
$$\frac{{\overline{μ}}_{v}}{{\overline{μ}}_{s}}$$
calculated for a smaller partition is greater than
$$\frac{{\overline{μ}}_{v}}{{\overline{μ}}_{s}}$$
calculated for a larger partition that contains the smaller partition.

4. The method of claim 2 further including:
following partitioning of the microarray data set, coalescing individual partitions that, when merged, have a combined \overline{μ}ₛ less than or equal to \overline{μ}ₛ for each individual partition and have a combined ratio
$$\frac{{\overline{μ}}_{v}}{{\overline{μ}}_{s}}$$
less than or equal to
$$\frac{{\overline{μ}}_{v}}{{\overline{μ}}_{s}}$$
for each individual partition.

5. The method of claim 2 further including recalculating feature positions taking into account detected block/zone misalignments.

6. The method of claim 2 wherein displacement vectors are calculated by matrix operations that determine a matrix
$$M = ( \begin{matrix}{m}_{xx} & {m}_{xy} & {O}_{x} \\ {m}_{yx} & {m}_{yy} & {O}_{y}\end{matrix} )$$
from a matrix containing the centroids of found features C and an inverse of an index matrix I as follows:
$$\begin{matrix}M = {CI}^{- 1} \\ where C = ( \begin{matrix}{x}_{1} & {y}_{1} \\ {x}_{2} & {y}_{2} \\ … & … \\ {x}_{n} & {y}_{n}\end{matrix} ) and I = ( \begin{matrix}1 & 1 & 1 & … & 1 \\ {c}_{1} & {c}_{2} & {c}_{3} & … & {c}_{n} \\ {r}_{1} & {r}_{2} & {r}_{3} & … & {r}_{n}\end{matrix} ) .\end{matrix}$$

7. A method comprising forwarding to a remote location one of:
a block/zone misalignment determined by the method of claim 1;
feature position data obtained by correcting for a block/zone misalignment determined by the method of claim 1; and
results obtained using a microarray data set and feature position data obtained by correcting for a block/zone misalignment determined by the method of claim 1.

8. A computer program implementing the method of claim 1 stored in a computer-readable medium.

9. A microarray data processing system that performs the method of claim 1.

10. A microarray data processing system comprising:
stored microarray data set;
a processor; and
a program executed by the processor that
calculates displacement vectors and displacement-vector-based metrics for the microarray data set comprising a digital image of a microarray and an initial feature indexing; and
partitions the microarray data set into smaller partitions until block/zone misalignments are each isolated in one or more partitions, as determined by displacement-vector-based metrics calculated for each partition.

11. The microarray data processing system of claim 11 wherein displacement-vector-based metrics include:
a vector sum of the displacement vectors for a partition within a microarray calculated by summing all displacement vectors dᵢ in the partition according to
$${μ}_{v} = {\sum }_{i = 1}^{n} {d}_{i}$$
where μᵥ is the a vector sum of the displacement vectors and n is the number of features;
a length of the vector sum of the displacement vectors within the partition calculated according to
\overline{μ}ᵥ=\sqrt{μᵥ·μᵥ}
where \overline{μ}ᵥ is the length of the vector sum of the displacement vectors; and
an average length of the displacement vectors dᵢ within the partition, calculated according to
$${\overline{μ}}_{s} = \frac{1}{n} {\sum }_{i = 1}^{n} \sqrt{{d}_{i} \cdot {d}_{i}}$$
where \overline{μ}ₛ is the average length of the displacement vectors.

12. The microarray data processing system of claim 11 wherein the computer program partitions the microarray data set into smaller partitions until block/zone misalignments are each isolated in one or more partitions by:
partitioning the microarray data into partitions until one of
\overline{μ}ₛ calculated for a partition falls below a threshold value,
\overline{μ}ₛ calculated for a final partition is less than or equal to \overline{μ}ₛ calculated for a larger partition that contains the final partition; and
$$\frac{{\overline{μ}}_{v}}{{\overline{μ}}_{s}}$$
calculated for a smaller partition is greater than
$$\frac{{\overline{μ}}_{v}}{{\overline{μ}}_{s}}$$
calculated for a larger partition that contains the smaller partition.

13. The microarray data processing system of claim 11 wherein the computer program, following partitioning of the microarray data set, coalesces individual partitions that, when merged, have a combined \overline{μ}ₛ less than or equal to \overline{μ}ₛ for each individual partition and have a combined ratio
$$\frac{{\overline{μ}}_{v}}{{\overline{μ}}_{s}}$$
less than or equal to
$$\frac{{\overline{μ}}_{v}}{{\overline{μ}}_{s}}$$
for each individual partition.

14. A method for correcting block/zone misalignments in a microarray data set including indexed features, the method comprising:
calculating displacement vectors for the features; and
identifying block/zone misalignments within the features in the microarray data set using the calculated displacement vector.

15. The method of claim 14 wherein identifying block/zone misalignments within the features in the microarray data set further comprises:
partitioning the microarray data set into smaller partitions until block/zone misalignments are each isolated in one or more partitions, as determined by displacement-vector-based metrics calculated for each partition.

16. The method of claim 14 wherein displacement vectors are calculated by matrix operations that determine a matrix
$$M = ( \begin{matrix}{m}_{xx} & {m}_{xy} & {O}_{x} \\ {m}_{yx} & {m}_{yy} & {O}_{y}\end{matrix} )$$
from a matrix containing the centroids of found features C and an inverse of an index matrix I as follows:
$$M = {CI}^{- 1}$$
$$where C = ( \begin{matrix}{x}_{1} & {y}_{1} \\ {x}_{2} & {y}_{2} \\ … & … \\ {x}_{n} & {y}_{n}\end{matrix} ) and I = ( \begin{matrix}1 & 1 & 1 & … & 1 \\ {c}_{1} & {c}_{2} & {c}_{3} & … & {c}_{n} \\ {r}_{1} & {r}_{2} & {r}_{3} & … & {r}_{n}\end{matrix} ) .$$

17. The method of claim 16 further including generating corrected feature positions for the features of the block/zone misalignments by determining an estimate for the correct positions of all features Fₐₗₗ within a partition, based on the matrix M determined using the observed positions of a set of known features, as follows:
Fₐₗₗ=MIₐₗₗ

18. A method comprising forwarding to a remote location one of:
feature position data obtained by correcting for a block or zone misalignment determined by the method of claim 14; and
results obtained using a microarray data set and feature position data obtained by correcting for a block or zone misalignment determined by the method of claim 14.

19. A computer program implementing the method of claim 14 stored in a computer-readable medium.

20. A microarray data processing system that performs the method of claim 14.
